(19) United States
(12) Patent Application Publication
(10) Pub. No.: US 2019/0159732 A1
(43) Pub. Date: May 30, 2019

(54) WEARABLE DEVICE AND ASSOCIATED METHOD

(71) Applicant: PixArt Imaging Inc., Hsin-Chu City (TW)

(72) Inventors: Hsiu-Ling Yeh, Hsin-Chu City (TW); Yung-Chang Lin, Hsin-Chu City (TW); Shin-Lin Wang, Hsin-Chu City (TW)

(21) Appl. No.: 15/826,720

(22) Filed: Nov. 30, 2017

(51) Int. Cl. A61B 5/00; A61B 5/024

(52) U.S. Cl. CPC A61B 5/7203; A61B 5/02427; A61B 2562/185; A61B 5/681; A61B 2562/0233; A61B 5/02438

(57) ABSTRACT

A wearable device includes: a light source, a sensor and a processor. The light source selectively operates in an illuminating mode or a non-illuminating mode, and generates an auxiliary light passing through a physical body in the illuminating mode. The sensor captures detecting images from the physical body, wherein the detecting images include at least one illuminating image captured while the light source is in the illuminating mode, at least one pre-illuminating image captured before the illuminating image is captured while the light source is in the non-illuminating mode, and at least one post-illuminating image captured after the illuminating image is captured while the light source is in the non-illuminating mode. The processor generates physiological information of the physical body according to the illuminating image, the pre-illuminating image and the post-illuminating image.

### BACKGROUND OF THE INVENTION

### 1. Field of the Invention

The present invention relates to a wearable device, and more particularly, to a wearable device capable of reducing the influence of ambient light in order to detect physiological information.

### 2. Description of the Prior Art

A wearable device applying photoplethysmography techniques to detect physiological information of a user (for example, heart rate) must be tightly attached to the user (for example, by the wrists); otherwise, the detected physiological information will not be 100% correct due to the influence of ambient light. Therefore, a novel design to reduce the influence of the ambient light is desired.

### SUMMARY OF THE INVENTION

One of the objectives of the present inventions is to provide a wearable device and an associated method to reduce the influence of ambient light.

According to an embodiment of the present invention, a wearable device is disclosed, comprising: a light source, a sensor and a processor. The light source selectively operates in an illuminating mode or a non-illuminating mode. In the illuminating mode, the light source generates an auxiliary light passing through a physical body. The sensor is arranged to capture detecting images from the physical body, wherein the detecting images comprise at least one illuminating image captured while the light source is in the illuminating mode, at least one pre-illuminating image captured before the illuminating image is captured while the light source is in the non-illuminating mode, and at least one post-illuminating image captured after the illuminating image is captured while the light source is in the non-illuminating mode. The processor is coupled to the sensing circuit, and is arranged to generate physiological information of the physical body according to the illuminating image, the pre-illuminating image and the post-illuminating image.

According to an embodiment of the present invention, a detecting method employed by a wearable device is disclosed, comprising: controlling a light source of the wearable device to selectively operate in an illuminating mode or a non-illuminating mode; in the illuminating mode, generating, by the light source, an auxiliary light passing through a physical body; capturing detecting images from the physical body, wherein the detecting images comprise at least one illuminating image captured in the illuminating mode, at least one pre-illuminating image captured before the illuminating image is captured while in the non-illuminating mode, and at least one post-illuminating image captured after the illuminating image is captured while in the non-illuminating mode; and generating physiological information of the physical body according to the illuminating image, the pre-illuminating image and the post-illuminating image.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram illustrating a wearable device attached to a user according to an embodiment of the present invention.

FIG. 2 is a diagram illustrating the wearable device of the embodiment of FIG. 1.

FIG. 3 is a diagram illustrating a time line of operating in the illuminating mode and the non-illuminating mode according to an embodiment of the present invention.

### DETAILED DESCRIPTION

Certain terms are used throughout the description and following claims to refer to particular components. As one skilled in the art will appreciate, manufacturers may refer to a component by different names. This document does not intend to distinguish between components that differ in name but not function. In the following description and in the claims, the terms “include” and “comprise” are used in an open-ended fashion, and thus should not be interpreted as a close-ended term such as “consist of”. Also, the term “couple” is intended to mean either an indirect or direct electrical connection. Accordingly, if one device is coupled to another device, that connection may be through a direct electrical connection, or through an indirect electrical connection via other devices and connections.

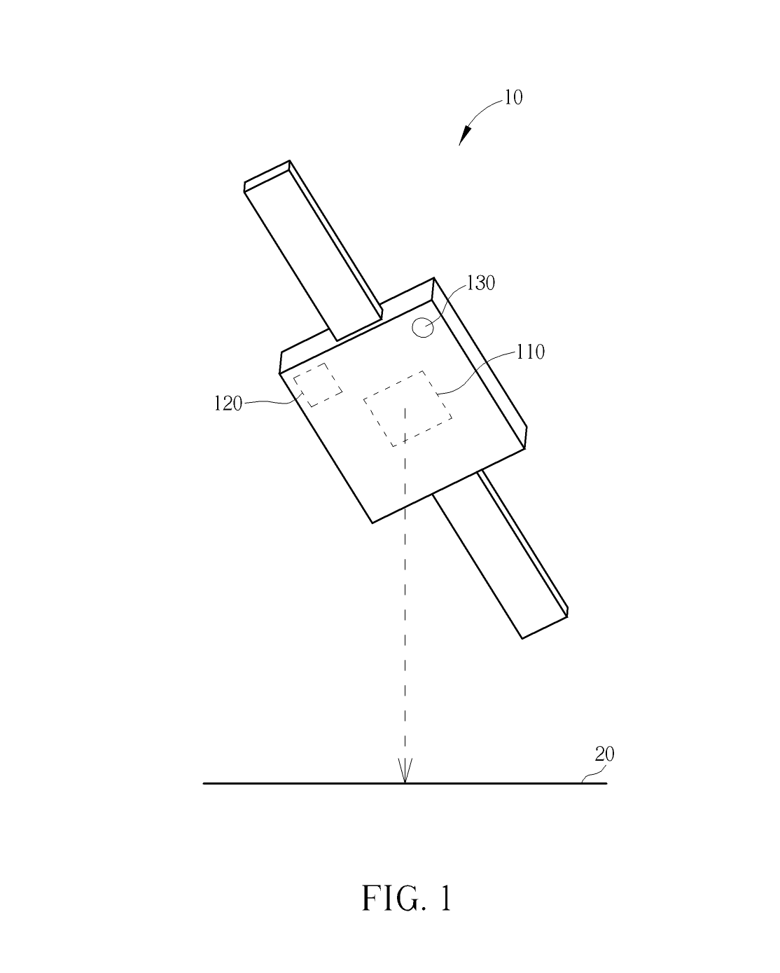

FIG. 1 is a diagram illustrating a wearable device 10 attached to a user 20 according to an embodiment of the present invention. It should be noted that the wearable device 10 depicted in FIG. 1 is a watch-shaped device wrapped around a wrist of the user 20; however, the wearable device 10 disclosed by the present invention is not limited to be a watch type device, and can also be a ring, earring, a pair of glasses, an armband etc. for detecting physiological information (e.g. heart rate) of the user 20. In the following paragraphs, the wearable device 10 is the watch-shaped device illustrated in FIG. 1.

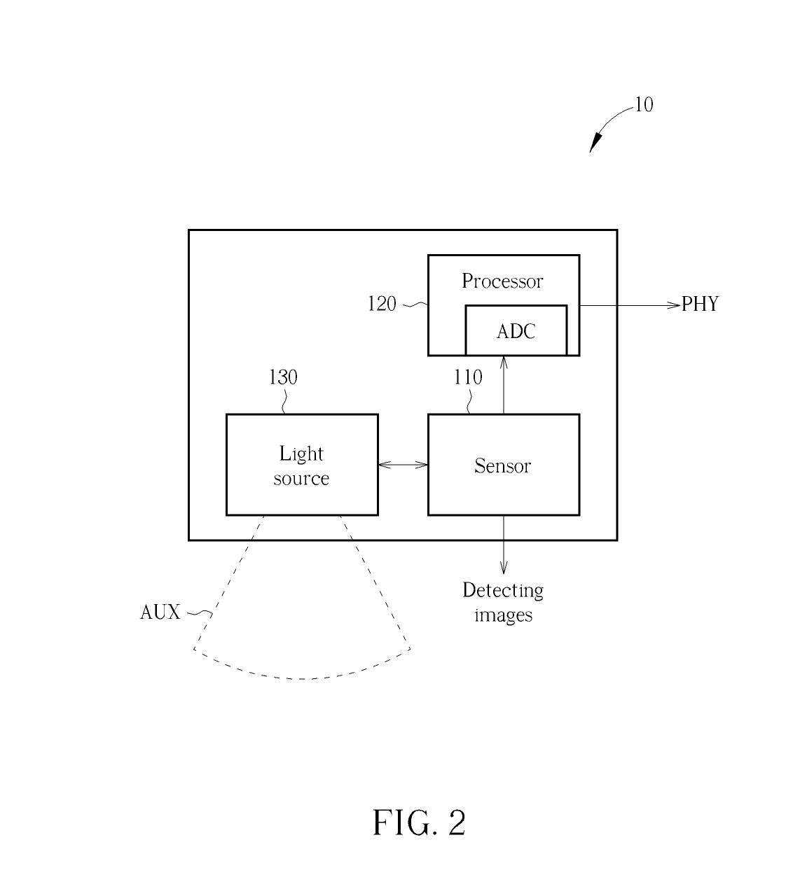

FIG. 2 is a diagram illustrating the wearable device 10 of the embodiment of FIG. 1. As shown in FIG. 2, the wearable device 10 comprises a sensor 110, a processor 120 and a light source 130. The light source 130 selectively operates in an illuminating mode and a non-illuminating mode, wherein in the illuminating mode the light source 130 provides an auxiliary light AUX passing through the body of the users 20 and does not provide the auxiliary light AUX in the non-illuminating node. The light source 130 while in the illuminating mode could emit light only when the sensor 110 capturing images. In this embodiment, the light source 130 may be implemented by a light emitting diode (LED). In this embodiment, the light source 130 alternatingly operates in the illuminating mode and the non-illuminating mode, i.e. the light source 130 repeatedly and regularly provides the auxiliary light AUX, wherein the lengths of operating in the illuminating mode and the non-illuminating mode could be equal and fixed. This is not, however, a limitation of the present invention. In other embodiments, the light source 130 may operate in the illuminating mode randomly, and the lengths of operating in the illuminating mode and the non-illuminating mode are not limited to be equal or fixed.

In this embodiment, the sensor 110 may be a camera for applying the photoplethysmography technique to detect physiological information, e.g. heart rate, of the user 20 by capturing detecting images of the user 20. The detecting images comprise illuminating images IMA₁-IMAᵢ captured in the illuminating mode (i.e. when the auxiliary light AUX is provided), pre-illuminating images PreIMA₁-PreIMAⱼ captured before the illuminating images IMA₁-IMAᵢ are captured while in the non-illuminating mode, and post-illuminating images PostIMA₁-PostIMAₖ captured after the illuminating images IMA₁-IMAᵢ are captured while in the non-illuminating mode, wherein j and k can be any positive integers. When i is 1, only one illuminating image (i.e. the illuminating image IMA₁) is captured. When j is 1, only one pre-illuminating image (i.e. the pre-illuminating image PreIMA₁) is captured. When k is 1, only one post-illuminating image (i.e. the post-illuminating image PostIMA₁) is captured. The number of captured detecting images is not a limitation of the present invention. Each of the detecting image could be provided as a 2D information (including X*Y pixel data) or a statistic information (such as intensity distribution or color distribution in 1D or 2D direction) of the 2D information.

The sensor 110, for sensing purposes, is preferably installed on a bottom surface of the wearable device 10 which attaches to the user's skin for higher accuracy, as shown in FIG. 1. This is only for illustrative purposes, and not a limitation of the present invention. The location of the sensing circuit 110 is based on the designer's consideration.

The processor 120 is arranged to process the detecting images captured by the sensor 110 to generate physiological information PHY which may be shown on a display (not shown in FIG. 2) of the wearable device 20 to inform the user 20. More specifically, the processor 120 transforms each of the detecting images captured by the sensor 110 into corresponding raw data which may be represented by a detected data, wherein the detected data may comprises a plurality of sub values and each sub value corresponding to one pixel of the captured image or the detected data may comprises one statistic value (such as intensity average/summation of the detected image).

For example, the pre-illuminating image PreIMA₁ corresponds to a pre-illuminating detected data PreData₁, wherein the pre-illuminating detected data PreData₁ may include the influence of the ambient light, the illuminating image IMA₁ corresponds to an illuminating detected data Data₁, wherein the illuminating detected data Data₁ includes the influence of the ambient light and the auxiliary light AUX passing through the body of the user 20, and the post-illuminating image PostIMA₁ corresponds to a post-illuminating detected data PostData₁, wherein the post-illuminating detected data PostData₁ includes the influence of the ambient light. The processor 120 generates the physiological information PHY according to the pre-illuminating detected data, the illuminating detected data, and the post-illuminating values. It should be noted that that transformation may be done by an analog-to-digital converter (ADC) of the processor 120. This is only for illustrative purposes, however. The process of transforming a detecting image into raw data should be well-known to those skilled in the art.

In a brief example, the sensor 110 includes four pixels (ex: 2×2 sensor array). The detected data for each detecting images is an intensity summation of the four pixels (Data=Pixel₁+Pixel₂+Pixel₃+Pixel₄), wherein Pixel₁, Pixel₂, Pixel₃ and Pixel₄ are intensity values of each pixel in the detecting image.

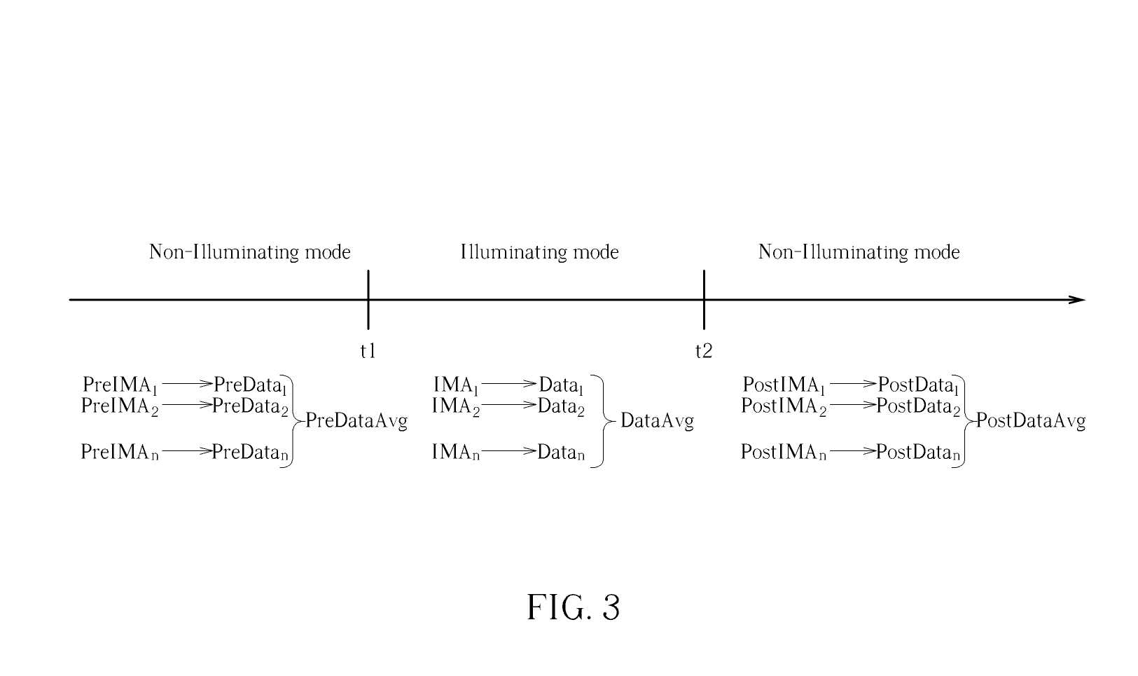

FIG. 3 is a diagram illustrating the time line of operating in the illuminating mode and the non-illuminating mode according to an embodiment of the present invention, wherein the light source 130 operates in the illuminating mode from t1 to t2, and operates in the non-illuminating mode before t1 and after t2 as shown in FIG. 3. The illuminating images IMA₁-IMAᵢ are captured by the sensor 110 from t1 to t2, the pre-illuminating images PreIMA₁-PreIMAⱼ are captured before t1, and the post-illuminating images PostIMA₁-PostIMAₖ are captured after t2. The processor 120 (or the ADC of the processor 120) generates the illuminating detected data Data₁-Dataᵢ corresponding to the illuminating images IMA₁-IMAᵢ, the pre-illuminating detected data PreData₁-PreDataⱼ corresponding to the pre-illuminating images PreIMA₁-PreIMAⱼ, and the post-illuminating detected data PostData₁-PostDataₖ corresponding to the illuminating images PostIMA₁-PostIMAₖ.

When j is not 1, i.e. more than one pre-illuminating image is captured, the processor 120 may further generate an average pre-illuminating detected data PreDataAvg from the pre-illuminating detected data PreData₁-PreDataⱼ. When j is 1, the average pre-illuminating detected data PreDataAVG can be easily derived from the pre-illuminating detected data PreData₁. In addition, when k is not 1, i.e. more than one pre-illuminating image is captured, the processor 120 may further generate an average post-illuminating detected data PostDataAvg from the post-illuminating detected data PostData₁-PostDataₖ. When k is 1, the average post-illuminating detected data PostDataAvg can be easily derived from the post-illuminating detected data PostData₁. Likewise, when i is not 1, i.e. more than one illuminating image is captured, the processor 120 may further generate an average illuminating detected data DataAvg from the illuminating detected data Data₁-Dataᵢ. When i is 1, the average illuminating detected data DataAvg can be easily derived from the illuminating detected data Data₁. To reduce the influence of the ambient light, the processor 120 generates an output detected data OutData by subtracting an average of the average pre-illuminating detected data PreDataAvg and the average post-illuminating detected data PostDataAvg from the average illuminating detected data DataAvg which can be represented by the following equation:

OutData=DataAvg−(PreDataAvg+PostDataAvg)/2.

Considering that the influence of the ambient light can be regarded as linear in a very short period, applying the above equation can effectively reduce the influence of the ambient light from the average illuminating detected data DataAvg, so that the output detected data OutData will only contain the influence of the auxiliary light AUX passing through the body of the user 20. In this way, the physiological information PHY generated by the processor 120 according to the output detected data OutData can be more accurate. It should be noted that the output detected data Outdata may be directly or indirectly regarded as the physiological information PHY (e.g. heart rate); for example, the output detected data Outdata may further be transformed into the heart rate of the user via some specific operations which will not be discussed in the present invention.

Briefly summarized, the present invention proposes a wearable device and an associated method to reduce the influence of ambient light by capturing illuminating images in the illuminating mode, pre-illuminating images and post-illuminating images in the non-illuminating mode, and subtracting the influence of the ambient light of the pre-illuminating images and the post-illuminating images from the illuminating images to assure high accuracy of the physiological information.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A wearable device, comprising:
a light source, selectively operating in an illuminating mode or a non-illuminating mode, wherein the light source generates an auxiliary light passing through a physical body in the illuminating mode;
a sensor, arranged to capture detecting images from the physical body, wherein the detecting images comprise at least one illuminating image captured while the light source is in the illuminating mode, at least one pre-illuminating image captured before the at least one illuminating image is captured while the light source is in the non-illuminating mode, and at least one post-illuminating image captured after the at least one illuminating image is captured while the light source is in the non-illuminating mode; and
a processor, coupled to the sensing circuit, wherein the processor is arranged to generate a physiological information of the physical body according to the at least one illuminating image, at least one pre-illuminating image and the at least one post-illuminating image.

2. The wearable device of claim 1, wherein the processor is further arranged to derive at least one pre-illuminating detected data from the at least one pre-illuminating image, at least one illuminating detected data from the at least one illuminating image, and at least one post-illuminating detected data from the at least one post-illuminating image, and generate an output detected data according to the at least one pre-illuminating detected data, the at least one illuminating detected data, and the post-illuminating detected data, wherein the physiological information is generated according to the output detected data.

3. The wearable device of claim 2, wherein the output detected data is generated by subtracting an average of the at least one pre-illuminating detected data and the at least one post-illuminating detected data from the illuminating detected data.

4. The wearable device of claim 2, wherein the at least one pre-illuminating detected data comprises a plurality of pre-illuminating detected data, the processor further generates an average pre-illuminating detected data according to the plurality of pre-illuminating detected data, and the output detected data is generated according to the average pre-illuminating detected data, the illuminating detected data and the at least one post-illuminating detected data.

5. The wearable device of claim 4, wherein the output detected data is generated by subtracting an average of the average pre-illuminating detected data and the at least one post-illuminating detected data from the illuminating detected data.

6. The wearable device of claim 2, wherein the at least one post-illuminating detected data comprises a plurality of post-illuminating detected data, the processor further generates an average post-illuminating detected data according to the plurality of post-illuminating detected data, and the output detected data is generated according to the at least one pre-illuminating detected data, the illuminating detected data and the average post-illuminating detected data.

7. The wearable device of claim 6, wherein the output detected data is generated by subtracting an average of the at least one pre-illuminating detected data and the average post-illuminating detected data from the illuminating detected data.

8. The wearable device of claim 1, wherein the light source operates alternately in the illuminating mode and the non-illuminating mode, the sensing circuit captures one detecting image each time the illuminating mode is on, and captures one detecting image each time the non-illuminating mode is on.

9. The wearable device of claim 1, wherein the processor comprises an analog-to-digital converter (ADC), and the output detected data is derived from an ADC output of the ADC.

10. The wearable device of claim 1, wherein the light source comprises at least one light emitting diode (LED).

11. A detecting method employed by a wearable device, comprising:
controlling a light source of the wearable device to selectively operate in an illuminating mode or a non-illuminating mode;
in the illuminating mode, generating, by the light source, an auxiliary light passing through a physical body;
capturing detecting images from the physical body, wherein the detecting images comprise at least one illuminating image captured in the illuminating mode, at least one pre-illuminating image captured before the at least one illuminating image is captured while in the non-illuminating mode, and at least one post-illuminating image captured after the at least one illuminating image is captured while in the non-illuminating mode; and
generating a physiological information of the physical body according to the at least one illuminating image, the at least one pre-illuminating image and the at least one post-illuminating image.

12. The detecting method of claim 11, wherein the physiological information comprises at least one pre-illuminating detected data corresponding to the at least one pre-illuminating image, at least one illuminating detected data corresponding to the at least one illuminating image, and at least one post-illuminating detected data corresponding to the at least one post-illuminating image, and the method further comprises:
generating an output detected data according to the at least one pre-illuminating detected data, the at least one illuminating detected data, and the post-illuminating detected data.

13. The detecting method of claim 12, wherein the output detected data is generated by subtracting an average of the at least one pre-illuminating detected data and the at least one post-illuminating detected data from the illuminating detected data.

14. The detecting method of claim 12, wherein the at least one pre-illuminating detected data comprises a plurality of pre-illuminating detected data, and generating the output detected data according to the at least one pre-illuminating detected data, the illuminating detected data, and the at least one post-illuminating detected data comprises:
generating an average pre-illuminating detected data according to the plurality of pre-illuminating detected data; and
generating the output detected data according to the average pre-illuminating detected data, the illuminating detected data and the at least one post-illuminating detected data.

15. The detecting method of claim 14, wherein generating the output detected data according to the average pre-illuminating detected data, the illuminating detected data and the at least one post-illuminating detected data comprises:
generating the output detected data by subtracting an average of the average pre-illuminating detected data and the at least one post-illuminating detected data from the illuminating detected data.

16. The detecting method of claim 12, wherein the at least one post-illuminating detected data comprises a plurality of post-illuminating detected data, and generating the output detected data according to the at least one pre-illuminating detected data, the illuminating detected data, and the at least one post-illuminating detected data comprises:
generating an average post-illuminating detected data according to the plurality of post-illuminating detected data; and
generating the output detected data according to the at least one pre-illuminating detected data, the illuminating detected data and the average post-illuminating detected data.

17. The detecting method of claim 16, wherein generating the output detected data according to the at least one pre-illuminating detected data, the illuminating detected data and the average post-illuminating detected data comprises:
generating the output detected data by subtracting an average of the at least one pre-illuminating detected data and the average post-illuminating detected data from the illuminating detected data.

18. The detecting method of claim 12, wherein the illuminating mode and the non-illuminating mode are alternately on, and one detecting image is captured each time the illuminating mode is on, and one detecting image is captured each time the non-illuminating mode is on.
